(19) United States
(12) Patent Application Publication
(10) Pub. No.: US 2007/0261176 A1
(43) Pub. Date: Nov. 15, 2007

(54) Apparatus and method for washing fibers

(76) Inventor: Dawin W. Stone, Ava, MO (US)

Correspondence Address: ROBERT J. LAUF, 998 W. OUTER DRIVE, OAK RIDGE, TN, 37830, US

(21) Appl. No.: 11/434,594

(22) Filed: May 15, 2006

(51) Int. Cl. A01H 5/02

(52) U.S. Cl. 8/148

(57) ABSTRACT

An apparatus for washing or otherwise wet-processing natural fibers in loose or “bulk” form comprises a generally cylindrical bag having cinch-type closures at both ends. The bag is doubled upon itself to form a generally annular cavity for holding the fibers. The inner cinch secures the first opening of the bag above the agitator of a conventional washing machine. After the fibers are placed into the bag, the outer cinch is secured, also above the agitator, thereby containing the fibers during selected washing or rinsing operations. The mesh size of the bag is selected to provide adequate fluid flow through the fibers while minimizing loss of the fibers or clogging of the plumbing. The two cinches are preferably tied together so that the bag and contents may be easily handled as a unit and hung as a unit for air drying.

### BACKGROUND OF THE INVENTION

1. Field of the Invention

The invention pertains to apparatus and methods for washing fibers and more particularly for apparatus and methods suitable for washing bulk natural fibers in a conventional washing machine.

2. Description of Related Art

All raw protein-based fibers such as wool must be washed before further processing. Historically large vats, pans, or kettles were used to hold the heated water, lye soap, and stale urine in which the fibers were washed. Although the term “washing” is normally associated with vigorous scrubbing or stirring, with wool and other protein fibers it is essential washing only involves soaking; agitation is normally avoided because this would cause felting. The fibers are traditionally lifted from the wash water and put into another pot of similar temperature rinse water. This is repeated if necessary until the fibers are clean. When the fibers are clean they are strung out on rocks, bushes, fences, or racks to dry.

With the advent of the top-loading washing machine and new formulations of soap or detergent this process became somewhat easier. To prevent machine and pipe problems, loose fibers are put into commercial zippered laundry bags, or pillowcases which are secured with safety pins or tied closed with string. One problem with this approach is that the fibers can collect in the comers of the bag and they must therefore be dumped out to dry. Also, using multiple small bags makes it difficult to balance the load in the washing machine.

Various bags have been devised for washing small and/or fragile garments in a conventional washing machine. Such “lingerie bags” generally have a single closure and are constructed of various materials ranging from open mesh to substantially opaque cloth. Such bags serve two primary purposes. First, they protect delicate garments from wear and tear and keep items like straps from becoming entangled with the agitator or with other items being washed at the same time. Second, they protect the other garments from being snagged or otherwise damaged by hooks or other features on the lingerie. A garment washing bag having two openings is disclosed by Sabounjian in U.S. Pat. App. Pub. 20050129337 published on Jun. 16, 2005; it is contemplated in '337 that the spindle of the washer will pass through both of said openings and that the entire bag will be substantially submerged during the wash cycle. It is further contemplated in '337 that the contents of the bag will be subjected to a substantially normal “laundry” washing cycle.

Industrial-scale processing equipment and methods are generally unsuitable for the hobby farmer or fiber artisan, who would naturally wish to use available facilities such as a home washing machine. However, a conventional washing machine has some inherent drawbacks when used for loose, bulk fibers. First, some fiber is inevitably lost, and second, the “lost” fiber can clog the washer or the plumbing. Thus, at present there is a need for a simple way to wash natural fibers, particularly various kinds of wool, in a conventional washing machine while avoiding the aforementioned problems.

Objects and Advantages

Objects of the present invention include the following: providing an apparatus for washing natural fibers in their loose or bulk form; providing an apparatus for washing natural fibers in a conventional top-loading washing machine; providing an apparatus for washing natural fibers while minimizing fiber loss and/or clogging of the washing machine; providing an apparatus for washing natural fibers that is inexpensive, reusable, and requires very little storage space; providing a method for washing natural fibers in a conventional top-loading washing machine; and, providing a method for washing natural fibers that minimizes fiber loss and clogging of the washing machine. These and other objects and advantages of the invention will become apparent from consideration of the following specification, read in conjunction with the drawings.

### SUMMARY OF THE INVENTION

According to one aspect of the invention, an apparatus for washing fibers comprises:
a generally cylindrical bag, the bag comprising a mesh fabric with a mesh size smaller than about 2 mm and having cinchable closures at each end,
the bag being at least 1 meter in length and at least 1 meter in circumference,
the bag further being foldable upon itself to form a generally annular bag capable of containing fibers in loose form while both of the cinchable openings are closed above the agitator of a washing machine.

According to another aspect of the invention, a method for washing fibers comprises the steps of:
a. forming a generally cylindrical bag with cinchable closures on each end;
b. folding the bag upon itself to form a generally annular cavity with an inner and an outer cinchable closure;
c. placing the bag into a washing machine and securing the inner cinchable closure above the agitator of the washing machine;
d. placing the fibers into the annular cavity;
e. securing the outer cinchable closure above the agitator of the washing machine; and,
f. operating the washing machine over a selected portion of a selected operating cycle.

### BRIEF DESCRIPTION OF THE DRAWINGS

The drawings accompanying and forming part of this specification are included to depict certain aspects of the invention. A clearer conception of the invention, and of the components and operation of systems provided with the invention, will become more readily apparent by referring to the exemplary, and therefore non-limiting embodiments illustrated in the drawing figures, wherein like numerals (if they occur in more than one view) designate the same elements. The features in the drawings are not necessarily drawn to scale.

FIG. 1 is a schematic diagram of one embodiment of the apparatus of the present invention.

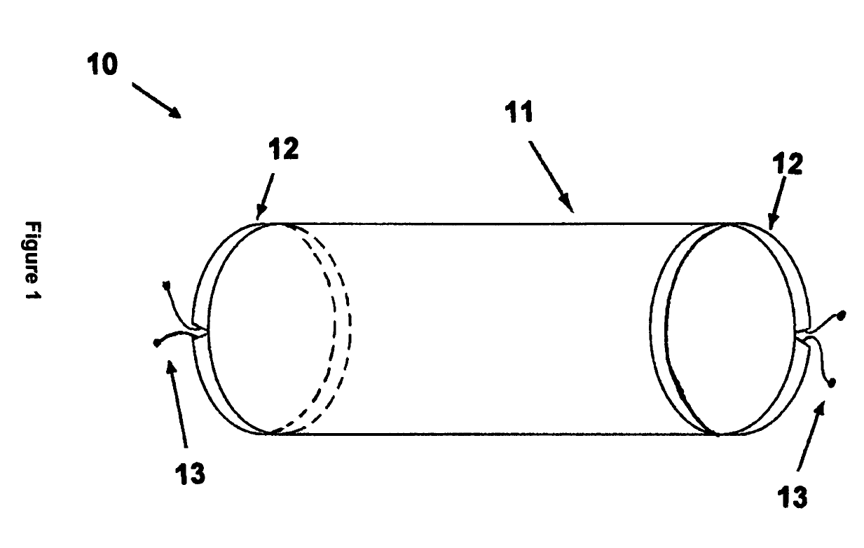

FIG. 2 is a schematic diagram in cross section of the invention folded upon itself to form a generally annular cavity.

FIG. 3 is a schematic diagram in cross section of the invention disposed within a conventional top loading washing machine. FIG. 3A shows the empty bag deployed in the washer with the first cinch closed; FIG. 3B shows bulk fiber in the open bag; and FIG. 3C shows the filled bag after closing the second cinch.

### DETAILED DESCRIPTION OF THE INVENTION

In its most general form, the invention comprises a bag for washing natural fibers in loose or bulk form in a standard top-loading washing machine, while minimizing loss of the fibers and/or clogging of the washing machine. It is particularly suited to animal fibers such as wool from sheep, Angora goat mohair, alpaca, llama, camel, musk ox, buffalo, etc. as well as more exotic fibers such as dog, cat, Angora rabbit, and so on. It can also be used when soaking handspun yam in preparation for setting the twist, soaking in the pre-dyeing preparation, or when intentionally felting an item by hand.

As will be shown in the following examples, the bag can constructed in a number of ways but a common feature is that the bag is generally cylindrical with a cinchable closure at both ends. The bag is of an appropriate size to fit within a conventional washing machine. To use the bag, it is first folded upon itself to form a generally annular bag with both cinchable openings at the top. The bag is placed into a washing machine and the inner cinchable opening is loosely secured above the agitator of the washing machine. The outer cinchable opening is spread open and the bulk fiber is evenly placed into the annular space. Then, the outer cinchable opening is secured, preferably also above the agitator, the cinches of both openings are preferably tied together, and the washing machine is operated over some selected portion of one of its washing cycles.

The bag and contents may be removed from the washer after a first selected operating cycle, and then placed back in the washer for a second selected operating cycle as will be discussed in the examples that follow. The bag may further be removed from the washer and hung by the cinched ends for air drying if desired.

### EXAMPLE 1

A bag 10 was constructed of ultra fine polyester netting 11 about 1.4×1.9 m. The two 1.4 m edges are brought together and seamed about 1 cm. Encasing the open ends in a stronger fabric makes a cord channel 12. Applicant has found that colorfast double fold bias quilt binding is particularly suitable. The edge binding is cut 1.9 m long and hemmed under about 1 cm on each end. One is sewn over each raw edge of the open cylinder; the two hemmed edges of the binding meet, but do not overlap. Two flexible cords 13 (preferably nylon) are cut about 2 m long, the ends are optionally melted with a flame to prevent raveling, and then one is threaded through each channel of the binding. An overhand knot secures the meeting cord ends.

Many types of fine mesh material are suitable for constructing the bag. Several suitable mesh fabrics include 0.25-mm polyester “No-See-Um”, 0.8-mm mosquito netting, and 840×1680 Denier nylon leno mesh [all available from American Home & Habitat, Route 4, Box 86, Squire, Mo. 65755]. Applicant has found that “No-See-Um” fabric is particularly suitable. (Through experimentation, Applicant previously determined that a conventional laundry bag with a mesh of about 6 mm was generally unsuitable for washing raw fibers because the fibers tend to protrude from such a large mesh, thereby defeating the purpose of the bag and that 2 mm is a reasonable upper limit on the suitable mesh size.)

### EXAMPLE 2

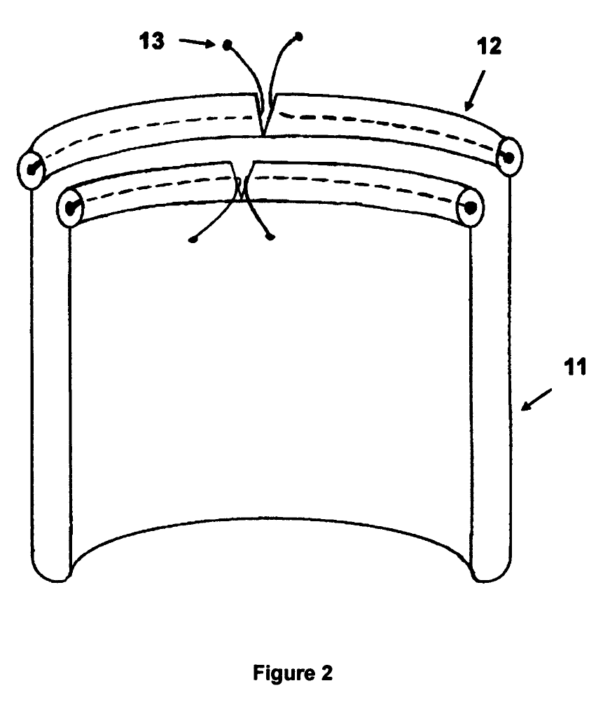

The inventive bag may be made in various sizes, but in any event will be generally larger than conventional laundry bags because it will be doubled upon itself in use as shown schematically in FIG. 2. The bag in the previous example when fully open was about 1.4 m long and about 2 m in circumference. As will be described in several of the following examples, the exact dimensions of the bag are not critical but are preferably of a convenient size for use in a selected top-loading washer. In most cases the bag will preferably be at least 1 m in length and less than about 2 m in length. If it is less than about 1 meter long, it will be too short after it is doubled up and the capacity of the bag will therefore be limited. If it is too long, it will be inconveniently long when the user removes it from the washing machine. The circumference of the bag is preferably larger than about 1 m and smaller than about 3 m. If the diameter is too small the capacity will be limited and if the diameter is too large it is wasteful of material.

The bag described in the foregoing examples is constructed of fine polyester netting, and because of the mass of wet product contained therein it is generally desirable to reinforce the open edges of the bag so that adequate strength is provided when lifting the wet product out of the washer. The edge binding described in Example 1 serves a strengthening function as well as a cinching function by providing the channel through which the cinching cord passes. It will be appreciated that other means may be employed for both the reinforcing function and the cinching function of the present invention.

### EXAMPLE 3

One suitable cinching approach is to place eyelets at various points along the open edges of the bag. A cinching cord may be looped back and forth through these eyelets. The eyelets are preferably reinforced with edge binding to prevent them from being pulled out of the netting during lifting or other manipulations of the filled bag.

It will be appreciated that the drawstrings may be secured in various conventional ways, such as tying a knot or using small cord locks or other fasteners as are well known in the art. Because it is often desirable to use the drawstrings not only to cinch the openings closed but also to hang the bag for air drying, it is preferable that the drawstrings be sufficiently strong to support the bag and its contents when wet.

### EXAMPLE 4

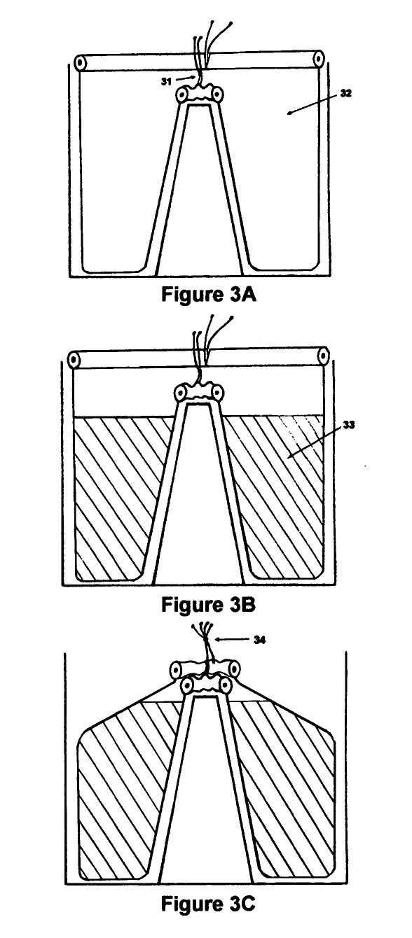

Referring to FIG. 3, the invention may be used as follows: As shown in FIG. 3A, one cord 31 is cinched to fit loosely above the agitator of a top-loading washing machine. The cord is tied into a knot and the central part of the bag is tucked down into the washer to form a generally annular cavity 32. The open end of the bag is spread outward to allow the fiber 33 to be placed evenly and loosely in the cavity 32 formed by the bag as shown schematically in FIG. 3B. The second opening is then cinched by cord 34 and tied as shown schematically in FIG. 3C. (In this process, cords 31 and 34 are preferably tied together as shown in FIG. 3C so that the entire bundle may be conveniently lifted as a unit.) The bundle is then temporarily removed from the machine. The washer is filled with hot water (typically 140° F.) and appropriate soap or detergent. The bag is then gently pressed into the water and allowed to remain submerged to soak for a selected time (preferably 3045 minutes). The machine cycle is stopped during this time so that the agitator will not operate, as this might cause felting of the fiber in the hot soapy water. The spin cycle is then activated (without spraying additional fresh water) to extract the wash water. The bag of fiber is then removed temporarily and, preferably, kept warm while preparing the rinse. To prevent shrinkage the rinse water is preferably the same temperature as the wash water in the previous step. It is frequently desirable to use a first rinse containing a small amount of vinegar, followed by a second rinse in pure water. The bag is then removed from the washer and hung by the two cinches to allow the fiber to drip dry.

### EXAMPLE 5

Skeins of handspun yam, whether single, or multiple ply, needs to have the twist equalized and set to become a good, non-twisting or undone yam when used for knitting, weaving, etc. This was traditionally accomplished by soaking the yam in very warm water, squeezing out the water, blotting the excess, then hanging it to dry. Using the inventive bag, the process can be accomplished by soaking in the warm water and then using the washer's spin cycle to remove the water. Arranging the skeins or yam around the agitator in the inventive bag makes this a simple process. It prevents the yams from becoming entangled and caught by the fins of the agitator.

### EXAMPLE 6

The dyeing process of protein fibers requires the fibers to be warm with a temperature similar to that of the dye bath, and damp with no excess water. The fibers may be loose to “dye-in-the-wool,” or a roving of carded and sectioned fibers, or as a spun yarn. Using the inventive bag, the fibers are soaked, drained, and spun to remove excess water. The fibers are then ready to be immediately put into the dye bath whether it is natural dyes from plants, animals (cochineal), mineral, or synthetic commercial dyes.

### EXAMPLE 7

Felting is generally the making of a fabric, a rug, or a molded item from loose protein fibers. Applicant has made felt from mounds of loose fibers laid at right angles several inches deep. (Previously knitted items may also be felted.) The inventive bag, flattened into a generally rectangular shape, contains the fibers within the two layers, (alternately, the bag may be used doubled over, depending on the size of the felted item being made) wraps the mound of fibers to confine, control, and compress them during the hand manipulation process. One method for making felt uses a lot of hot soapy water, rubbing, pounding, flipping the item over and over then rinsing with cold water. This is repeated until the desired thickness and dimensions are attained. Another method frequently used for making rugs is lightly sprinkling soapy lukewarm water over the mounds of fiber. The bag with the fibers somewhat compressed and beginning to adhere to each other (making felt) are then wrapped around a large diameter rod, which is rolled repeatedly on a floor or table until the felted fabric becomes very hard. Good felt is achieved when no fiber can be pulled free of the item, regardless of the felting method.

### EXAMPLE 8

The invention may further be used in various drying operations, where its design allows the bag to be hung by the knotted cords or flattened with the knotted cords loosened thereby forming a flat, generally rectangular “pouch”. The washed loose fibers, etc. can more quickly be dried by spreading them out and suspending them (preferably horizontally) across several parallel clotheslines appropriately spaced at a convenient height above the ground. The clothespins or clamps secure the bag to the multiple clotheslines and close the open ends.

Skilled artisans will appreciate that the inventive apparatus has significant differences compared to items such as lingerie bags: First, the function of a lingerie bag is different, viz., protecting delicate garments from wear and tear; it is intended to be used for washing things along with other items in a “normal” wash cycle. Second, the structure of a lingerie bag is different, viz., it has only one open end, a smaller overall size, and lack of means to attach it to the agitator. Third, the method of using a lingerie bag is different, viz., it is filled with lingerie, closed, added to the washer along with other items, and the washer is run through a normal wash cycle.

Several types of laundry bags are disclosed by Sabounjian in U.S. Pat. Appl. Pub. 2005/0129377. The bags in '337 have openings to allow the agitator of a washer to pass through. Two different geometric arrangements are shown, both of which have serious shortcomings which preclude their use for washing loose fibers. The first arrangement of '337, shown generally in FIGS. 14, has two cinchable ends designed to clasp the agitator, one near the bottom and the other near the top. Although the agitator in '337 is represented in FIG. 3 as a substantially smooth cylinder for most of its length, real agitators tend to be strongly tapered from bottom to top and further have pronounced ribs or fins to induce the desired agitation of the water and laundry. Thus, a cinch applied snugly to the surface of a conical body (as opposed to a cylindrical body) is inherently unstable and will tend to creep upward and become loose. Furthermore, because of the fins on real agitators, the resulting large gap between the cinch and the main body of the agitator would allow fibers to escape from the bag. Furthermore, because the two cinched ends in '337 are not tied together, the filled bag cannot be lifted from the washer as a unit and cannot be conveniently hung as a unit to dry. The second arrangement of '337, shown generally in FIGS. 5-8, has a central, cylindrical passage to accommodate the agitator and a cinchable opening on the outer surface through which the laundry items are loaded. This design also cannot be conveniently loaded uniformly with loose fibers and cannot be lifted from the washer as a unit. By contrast, Applicant's fiber washing bag is of such a size and shape that both cinches are closed above the agitator (and generally completely above the water) and the cinches can be tied together so that the bag and its contents may be lifted and handled as a unit. Sabounjian '337 further discloses a method of washing clothing using a substantially conventional wash cycle (specifically including rotation of the agitator). Applicant's method, by contrast, employs a substantially static soaking cycle without agitation in order to prevent felting of the loose fibers.

Skilled artisans will also appreciate that the invention provides significant benefits over methods currently used in the art of natural fiber processing. The single donut-shaped bag surrounding the agitator of a top-loading washing machine is significantly more convenient, because it is a single easy-to-manipulate bag, instead of multiple smaller bags, which can be difficult to balance. Applicant has found that when using several small bags the fibers tend to lump in corners and then they must be dumped out to dry. The single bag is sized to conveniently fill a top-loading washing machine tub, it has no comers, it is easy to insert and take out during the changing of the waters, and the entire bag with the fibers retained inside is easily hung as a unit to dry. During the drying time the bag can be fluffed and bounced to redistribute the fibers, if desired, to help speed the drying process.

As used herein, the term natural fiber encompasses any plant or animal-derived product that has at least some fibrous aspect or quality. Plant-derived (cellulose) materials include fibers such as cotton, flax, and hemp, and other products such as wood fibers, papyrus, bamboo, and the like. Animal-derived (protein) materials may include any type of wool, hair, fur, or like products obtained from mammals, as well as feathers, down, or similar products obtained from birds and fowl. Furthermore, it will be appreciated that the inventive process may be used to wash raw fiber materials, for example, raw wool that has just been sheared from the animal, as well as to wash, rinse, dye, felt, or otherwise treat fibrous materials that have undergone some earlier processing, such as threads and yams.

The terminology used by natural fiber artisans is not completely standardized. According to the Wool Products Labeling Act of 1939, “The term ‘wool’ means the fibers from the fleece of the sheep or lamb or hair of the Angora or Cashmere goat (and may include the so-called specialty fibers from the hair of the camel, alpaca, llama, and vicuna) . . . . ” However, hand spinners and fiber artists typically use the word “wool” to mean the fibers shorn once or twice a year from domesticated sheep or lambs. Wool from a sheep has natural wavy crimps, which varies from many for fine Merino wool to few with coarse carpet wools. Under a microscope wool has overlapping scales which connect only at the base to the fiber. The length and texture of the fiber varies with the sheep breed (there are hundreds of breeds worldwide) and the part of the body from where it grows. Hair from a fiber animal generally has no crimp. It may be coarse or fine depending upon the type of animal and the age of the animal (mohair from the young Angora goat is very silky but becomes coarser with each shearing as the animal ages). The fiber lengths also vary with the type of animal. Fur from a mammal is relatively short, fine, slippery and has no crimp. Down is the undercoat of fine, soft, short fibers of particular mammals. The term down is also used to denote the very soft, fluffy inner feather of some adult birds. All of the foregoing protein fibers may be processed using Applicant's invention.

Skilled artisans will appreciate that loose cellulose fibers are generally not washed before spinning. Boiling has formerly been used to set the twist of cotton, but modern practice is moving away from some traditional treatments. Also, many new commercial formulations of cellulose fibers are available for spinners, but they are “washed” in the commercial preparation for spinners. However, even in these applications, the inventive bag may be usefully employed for pre-dyeing preparation of cellulose fibers.

I claim:

1. An apparatus for washing fibers comprising:
a generally cylindrical bag, said bag comprising a mesh fabric with a mesh size smaller than about 2 mm and having cinchable closures at each end,
said bag being at least 1 meter in length and at least 1 meter in circumference,
said bag further being foldable upon itself to form a generally annular bag capable of containing fibers in loose form while both of said cinchable openings are closed above the agitator of a washing machine.

2. The apparatus of claim 1 wherein said mesh fabric comprises woven monofilament polyester having a mesh size from about 0.1 to 0.5 mm.

3. The apparatus of claim 1 wherein said cinchable closures comprise flexible cords contained within hems at each of said ends.

4. The apparatus of claim 3 wherein said hems are further reinforced with double fold bias quilt binding.

5. The apparatus of claim 1 wherein said cinchable closures comprise cords passing through eyelets.

6. The apparatus of claim 1 wherein said fiber is a protein fiber selected from the group consisting of: animal fur; sheep wool; Angora goat mohair; cashmere; buffalo; musk ox; camel; yak; alpaca; llama; dog fur; cat fur; Angora rabbit fur; poultry feathers; and poultry down.

7. The apparatus of claim 1 wherein said fiber is a cellulose fiber selected from the group consisting of: cotton fiber; hemp fiber; flax fiber; wood fiber; and papyrus.

8. A method for washing fibers comprising the steps of:
a. forming a generally cylindrical bag with cinchable closures on each end;
b. folding said bag upon itself to form a generally annular cavity with an inner and an outer cinchable closure;
c. placing said bag into a washing machine and securing said inner cinchable closure above the agitator of said washing machine;
d. placing said fibers into said annular cavity;
e. securing said outer cinchable closure above said agitator of said washing machine; and,
f. operating said washing machine over a selected portion of a selected operating cycle.

9. The method of claim 8 wherein said selected portion of said selected operating cycle comprises soaking in water at a selected temperature without agitation, followed by removal of said water by spinning.

10. The method of claim 8 further including the step of:
g. removing said bag from said washing machine and hanging said bag by said cinchable closures to air dry said fibers.

11. The method of claim 8 wherein said bag comprises a mesh fabric comprising woven monofilament polyester having a mesh size from about 0.1 to 0.5 mm.

12. The method of claim 8 wherein said wherein said fiber is a protein fiber selected from the group consisting of: sheep wool; Angora goat mohair; cashmere; buffalo; musk ox; camel; yak; alpaca; llama; dog fur; Angora rabbit fur; feathers; and down.

13. The method of claim 8 wherein said fiber is a cellulose fiber selected from the group consisting of: cotton fiber; hemp fiber; flax fiber; wood fiber; and papyrus.
